(19) United States
(12) Patent Application Publication
(10) Pub. No.: US 2022/0194829 A1
(43) Pub. Date: Jun. 23, 2022

(54) SYSTEM FOR MONITORING A BIOCHEMICAL PROCESS

(71) Applicant: COMMISSARIAT A L'ENERGIE ATOMIQUE ET AUX ENERGIES ALTERNATIVES, Paris (FR)

(72) Inventors: Régis PERRIER, Grenoble Cedex 09 (FR); Florian PORTE, Grenoble Cedex 09 (FR)

(73) Assignee: COMMISSARIAT A L'ENERGIE ATOMIQUE ET AUX ENERGIES ALTERNATIVES, Paris (FR)

(21) Appl. No.: 17/645,559

(22) Filed: Dec. 22, 2021

(30) Foreign Application Priority Data
Dec. 23, 2020 (FR) 20 14076

(51) Int. Cl. C02F 3/00; G01K 13/02; G01N 9/26; C12G 1/022; C12M 1/36; C12M 1/34

(52) U.S. Cl. CPC C02F 3/006; C02F 2209/24; G01N 9/26; C12G 1/0203; C12M 41/48; C12M 41/12; C12M 41/40; C12M 41/42; C02F 2209/02; C02F 2209/03; C02F 2209/38; C02F 2209/42; C02F 2209/05; C02F 2209/06; C02F 2209/04; C02F 2209/22; G01K 13/026

(57) ABSTRACT

The invention relates to a method and a system for in-situ monitoring of a biochemical process in a reactor comprising a vessel (5) intended to receive a liquid (7), said system comprising:
a measuring device (9) intended to be inserted floating into said vessel 5, said measuring device (9) being instrumented with sensors configured to take measurements relating to the biochemical process at successive instants and to transmit, at said successive instants, observation data representing said measurements; and
a control device (11) configured to control the regulation of the biochemical reactor (3) at said successive instants, according to said observation data received from the measuring device (9).

### TECHNICAL FIELD

The present invention relates to the field of monitoring biochemical processes.

### PRIOR ART

The regulation of a biochemical process can address many industrial applications in various fields such as, agri-food (wine, cider, beer, spirits, liquid foods . . . ), pharmaceuticals (medicines or food supplements . . . ), environmental industry (water analysis in treatment plants, methanisation . . . ), petrochemicals (biogas . . . ), electrochemicals, etc.

Biochemical processes involve the action of microorganisms on complex substrates (fruit juice, leachate, etc.). They are generally regulated by manual off-line analysis in industrial environments, such as the agri-food industry. Regulation in bioreactors by monitoring on-line control parameters is reserved for high added-value products, such as medical products.

The control parameters generally include temperature measurements, and possibly other additional measurements that may be application specific, such as acidity, turbidity, gas release, density, dissolved oxygen, etc.

In the case of alcoholic beverages, it is necessary to measure the temperature and density of a liquid in order to control the food quality, such as sugar and alcohol content. In order to produce medicines or analyse water in treatment plants, the measurements relate more generally to the pH, redox potential, conductivity and presence of oxygen and dissolved carbon dioxide, etc.

As concerns density measurement, there are manual densimeters that are widely used in various fields of activity. Their dimensions and weight define the range and accuracy of measurement. However, they require samples in order to perform off-line analyses and operations are completely manual.

There are electronic densimeters used to measure the oscillation frequency of U-shaped tubes that vary according to the mass of the liquid sampled. These devices can be used on-line, but are expensive and sensitive to certain disturbances (fouling, bubbles).

Alternatively, there are devices that determine the density of a liquid by measuring the refractive index. However, the measurement can be disturbed by the presence of gases (e.g., CO₂) or fouling of the sensor.

All of these devices can be used for on-line measurements in some cases, but not “in situ”. Nevertheless, there are ball-shaped stand-alone modules for measuring pH, conductivity, pressure and movement in a liquid. These devices can be freely circulated in bioreactors and can thus be used for in situ measurements. However, these modules are limited to very specific measurements and not suitable for measuring density. Additionally, data transmission is not easy from the liquid medium and moreover, their location in the liquid is unknown, thus resulting in a lack of spatial reference for measurements.

Furthermore, bioprocesses generally involve regulation of temperature, agitation, oxygenation and nutrient supply. The aim of determining these control parameters is to improve both product quality and yield, while optimising energy and nutrient supplies. This control generally relies on the expertise of the user (oenologist in the case of wine, microbiologist for pharmaceuticals . . . ) and can thus be extremely tedious and time-consuming.

The object of the present invention is to remedy the above-mentioned drawbacks by proposing an automatic and accurate adjustment of the control parameters of the desired product, using real-time and in situ monitoring of how the biochemical process evolves, while enabling accurate multi-parameter measurements by means of a single measuring device.

### SUMMARY OF THE INVENTION

The invention relates to a system for in-situ monitoring of a biochemical process in a reactor comprising a vessel intended to receive a liquid, said system comprising:
a measuring device intended to be inserted into said vessel, said measuring device being instrumented with sensors configured to take measurements relating to the biochemical process at successive instants and to transmit, at said successive instants, observation data representing at least the temperature and the density, and
a control device configured to control the regulation of the biochemical reactor at successive instants according to said observation data received from the measuring device.

Thus, the measuring device can be used to monitor the evolution of physical parameters and transmit them to the control device, such that the latter can control the reaction in the vessel through feedback and in real time. Thus, this system can be used to automatically control the biochemical process while reacting directly in real time in the event of detection of an anomaly in the process.

Advantageously, said measurements comprise measurements of temperature of the liquid and at least one other type of measurements among the following measurements: mechanical measurements of pressures and/or gas flow rate, and/or accelerations and/or buoyancy level, electrical measurements of voltages and/or currents and/or resonance frequencies, and optical measurements.

This creates multiple indicators for more efficient and more accurate feedback and control of the biochemical process.

According to one embodiment, the measuring device comprises a microprocessor configured to determine, at said successive instants, vectors of physical variables relating to the biochemical process according to the corresponding measurements made at said successive instants, the observation data transmitted by the measuring device to said control device comprising said vectors of physical variables and said corresponding measurements.

Thus, the measuring device processes the measurements to determine, over time, vectors of physical values of interest including density before transmitting them to the control device.

According to another embodiment, the observation data transmitted by the measuring device to said control device comprises said measurements. In this case, the control device is configured to determine, at said successive instants, vectors of physical variables relating to the biochemical process according to the corresponding measurements.

According to this embodiment, the measuring device transmits only the measurements and these are processed by the control device in order to determine the physical parameters of interest. This helps to minimise processing and power consumption in the measuring device.

Advantageously, each of said vectors of physical variables comprises a temperature variable, a liquid density variable determined from measurements of buoyancy levels and/or pressures, and at least one other variable from among the following variables: gas release determined from pressure measurements, electrical conductivity and/or permittivity of the liquid determined from electrical measurements, movement of the liquid determined from acceleration measurements, PH and/or redox potential determined from electrical measurements, dissolved oxygen and/or CO₂ determined from electrical measurements and/or optical measurements, optical absorption spectrum and/or rotatory power determined from optical measurements.

These physical indicators are used to detect with great accuracy the slightest anomaly in the biochemical process.

Advantageously, the control device is configured to control regulation of the biochemical reactor by at least one of the following actions: modification of the agitation speed, modification of the temperature, modification of the rate of oxygen supply, nutrient supply or other elements for activating or stabilising the biochemical process, supply of yeasts or bacterial strains.

Advantageously, the control device is configured to:
predict, at said successive instants, predictive vectors of physical variables according to said previous vectors of physical variables derived from the measurements,
predict, at said successive instants, anticipative measurements according to said predictive vectors of physical variables,
calculate, at said successive instants, measurement discrepancies between the anticipative measurements and the corresponding actual measurements,
correct, at said successive instants, vectors of physical variables according to said measurement discrepancies,
determine, at said successive instants, vectors of regulation actions according to said vectors of corresponding physical variables, and
control regulation of the biochemical reactor by triggering, at said successive instants, regulation actions based on said vectors of regulation actions.

This helps to accurately determine, for each time instant, a series of actions with full knowledge of the physical variables observable by via the sensors of the measuring device.

Advantageously, the control device is configured to determine the vectors of regulation actions according to the predictive vectors of physical variables in the event of failure of the measuring device.

This helps to continue the regulation process in the event of a momentary malfunction of the measuring device.

Advantageously, the control device is configured to:
predict each predictive vector of physical variables using a first temporal function f defining values of physical variables with full knowledge of their values at a previous time instant, said first function f being predetermined by a standard Markov model of the order at least 1,
predict each anticipative measurement using a second function g associating the measurements performed by the measuring device with the physical variables at a given time instant, said second function g being predetermined by equations correlating the measurements with the physical variables, and
determine each vector of control actions using a third function h defining a correspondence between the actions to be carried out and the values of the physical variables at a given time instant, said third function h being predetermined by a pre-selection of action triggering thresholds.

These functions can be pre-adapted to be used in a simple manner to monitor any biochemical reaction.

Advantageously, the monitoring system comprises a database built during a regulation learning phase comprising correspondence data between measurements m_{1:T} performed by the measuring device and actions a_{1:T} performed in the vessel, said correspondence data being generated automatically by a learning process, and at least one of said first, second and third functions is determined from said correspondence data acquired from said database.

Thus, in the event where at least one of the three functions is unknown, it can be learned automatically with full knowledge of a complete time series of a biochemical reaction previously performed by experts in the biochemical process.

Advantageously, the control device is further configured to build a property learning model defining correlations associating properties p of the final product according to the corresponding physical θ_{1:T} and characteristic c variables. Knowledge of the property learning model helps to obtain a product with properties p, knowing its characteristics c, through the regulation of its physical variables θ_{1:T} during the biochemical reaction process. Indeed, with knowledge of the model that correlates the physical variables and the characteristics to the properties of the product on the one hand, and the input characteristics c (e.g. in the case of wine it could be the grape variety) on the other hand, the control device during the biochemical reaction can force the physical variables θ_{1:T} to follow a certain profile in order to obtain desired properties p at the outlet. In other words, the control device is used to adapt the characteristics of the elements placed in the vessel and to regulate the evolution of physical variables of biochemical reactions according to characteristics of the elements placed in the vessel, in order to obtain the desired specific properties of the product at the outlet of the reactor.

Advantageously, the control device is configured to determine said correlations by estimating a learning function using a statistical model of the polynomial regression type, or kernel model, or neural network.

Thus, the learning function helps to understand the influence of characteristics of reacted elements, or of evolution of the biochemical process in the properties obtained on the product.

Advantageously, the measuring device comprises at least one temperature probe, and a differential pressure sensor comprising two connected tubes of different lengths.

Advantageously, the measuring device is integrated into a float comprising:
at least one temperature probe,
one differential pressure sensor comprising two tubes of different lengths partly immersed in the liquid,
at least two electromagnetic, electrochemical or optical sensors comprising sensitive parts mounted on the walls of the float close to the liquid,
a movement sensor configured to measure movements of the float,
a wireless communication module comprising an antenna arranged on the upper part of the float, said communication module having been intended to transmit data relating to measurements performed by the sensors,
a power management module comprising a power supply means arranged in the lower part of the float, and
an electronic circuit connected to all the sensors and electronic elements integrated into the float, said electronic circuit comprising a microprocessor intended to manage the acquisition of all the sensors, a first processing level and data transmission.

The float is very compact and can be installed very simply in a broad variety of vessels, regardless of their filling level. Additionally, it allows for very accurate density measurement both in a transient state at the start of the process and in a steady state. Moreover, with the antenna arranged in the emerging part of the float, data can thus be transmitted simply, regularly and accurately.

Advantageously, the float comprises a calibration and inductive recharging module intended to calibrate the float and to remotely recharge the power supply means integrated into the float, said calibration and inductive recharging module being comprised in a protective case of the float.

Thus, the float is completely self-contained while allowing for fusion and synthesis of various measurements in a single embedded device.

The invention also relates to a method for in-situ monitoring of a biochemical process in a reactor comprising a vessel intended to receive a liquid, said method comprising the following steps:
take measurements relating to the biochemical process at successive instants,
transmit, at said successive instants, observation data representing at least the temperature and the density, and
control, at said successive instants, regulation of the biochemical reactor according to said observation data.

Other characteristics and advantages of the invention will become apparent in the non-limiting detailed description hereunder.

### BRIEF DESCRIPTION OF THE FIGURES

By way of non-limiting examples, embodiments of the invention will now be described, making reference to the annexed drawings wherein:

FIG. 1 very schematically illustrates a system for in-situ monitoring of a biochemical process in a biochemical reactor, according to one embodiment of the invention;

FIG. 2A and

FIG. 2B illustrate an algorithm for controlling the biochemical reactor, according to a preferred embodiment of the invention;

FIG. 3 is a directed graph representing a property learning algorithm of the compound at the outlet of the biochemical reactor, according to a preferred embodiment of the invention;

FIG. 4 very schematically illustrates a system for in-situ monitoring of a biochemical fermentation process in a reactor, according to a preferred embodiment of the invention;

FIG. 5 very schematically illustrates a method for in-situ monitoring of the biochemical fermentation process in relation to the system on FIG. 4;

FIG. 6 very schematically illustrates a system for in-situ monitoring of a biochemical process in a biochemical reactor, according to a preferred embodiment of the invention;

FIG. 7 very schematically illustrates an instrumented float, according to a preferred embodiment of the invention; and

FIG. 8 very schematically illustrates the arrangement of an electromagnetic capacitive sensor on the side surface of the float, according to the embodiment on FIG. 7.

### DESCRIPTION OF EMBODIMENTS

The principle of the invention is to automatically control a biochemical process taking place in a vessel using measurements performed in situ.

Thus, the invention proposes to adjust the control parameters according to the characteristics of the raw material and the properties of the desired product throughout the biochemical process according to how it evolves. Additionally, such control is performed by way of real-time and in situ monitoring of how the biochemical process evolves, while enabling accurate multi-parameter measurements by means of a single measuring device.

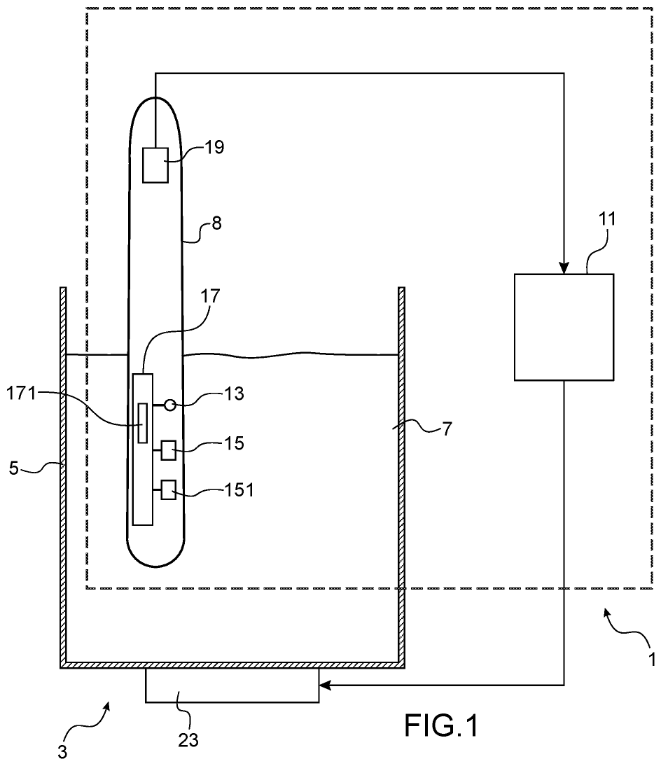

FIG. 1 very schematically illustrates a system 1 for in-situ monitoring of a biochemical process in a biochemical reactor 3, according to one embodiment of the invention.

The biochemical reactor 3 comprises a vessel 5 intended to receive a liquid substrate 7 on which a biochemical process involving the action of microorganisms has to be performed. Furthermore, the monitoring system 1 comprises a measuring device 8 and a control device 11.

In one embodiment, the measuring device 8 can be mounted on the top of the vessel 5, for instance, while being partially immersed in the liquid. This embodiment is advantageous in the case of small reactors.

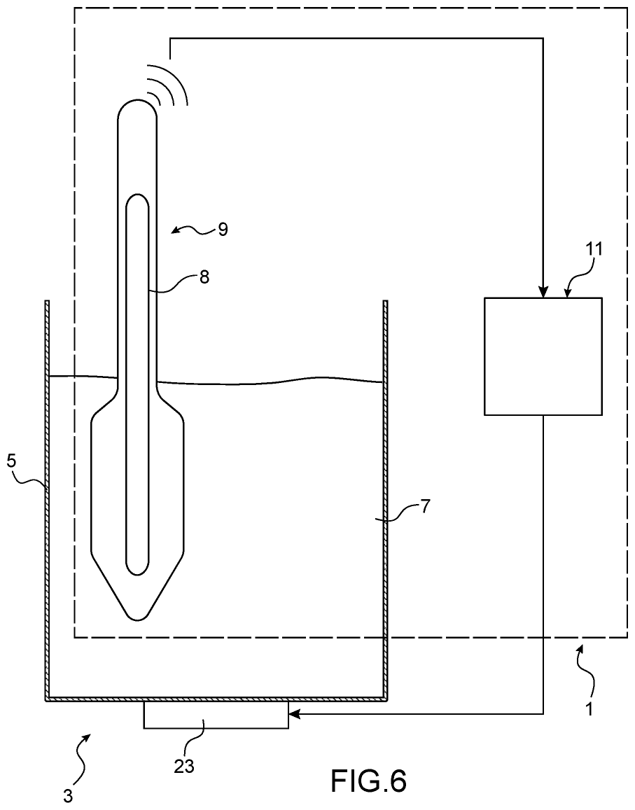

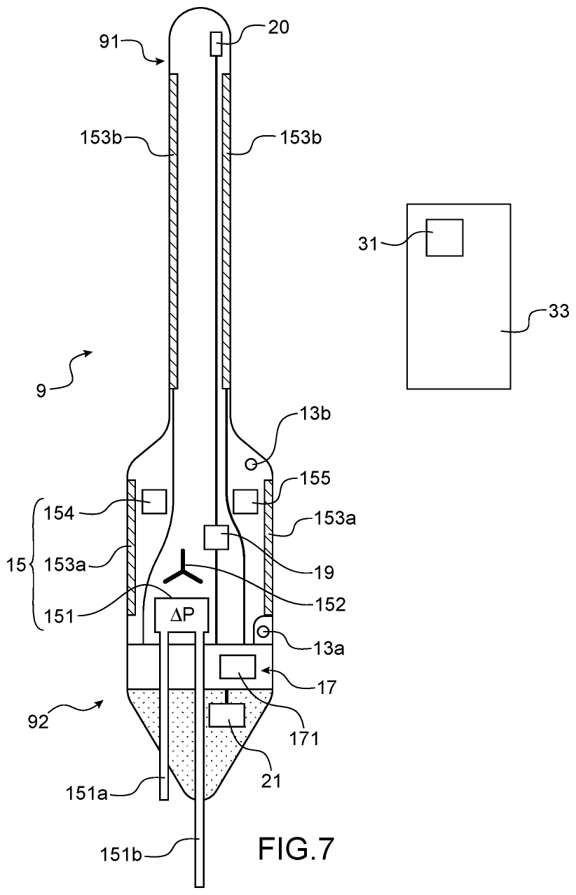

In another embodiment, the measuring device 8 may be embedded or integrated into a float intended to be inserted by floating partially immersed in the liquid 7 contained in the vessel 5 (cf. FIGS. 6 and 7). This embodiment is advantageous for all kinds of reactors, particularly for large vessels with a variable filling level.

The measuring device 8 is equipped with measuring sensors configured to perform measurements relating to the biochemical process directly on the liquid 7 at successive instants, and to transmit, at successive instants, observation data representative of these measurements to the control device 11.

The control device 11 (e.g., a computer or a microcomputer) is configured to receive observation data transmitted by the measuring device 8 and to control, at successive instants, regulation of the biochemical reactor 3 according to observation data received from the measuring device 8.

This monitoring system 1 can thus be used to automatically control the biochemical process while reacting directly in real time in the event of detection of an anomaly in the process.

Advantageously, the measuring device 8 incorporates at least one temperature probe 13 for measuring the temperature of the liquid 7, and at least one other measurement sensor 15 for measuring the density of the liquid and/or the gas flow rate.

In addition to the sensors for measuring the temperature 3 and density 15 of the liquid, the measuring device 8 comprises at least one other measurement sensor 151 for performing mechanical, electrical, optical or chemical measurements. Mechanical measurements comprise measurements of pressures in the liquid and/or of gas flow and/or of acceleration movements within the liquid medium. Electrical measurements comprise measurements of voltages and/or currents at one or more frequencies associated with specific electrodes arranged in the measuring device 8, and/or measurements of resonance frequencies of oscillators integrated into the measuring device 8. Optical measurements comprise measurements of light intensities at one or more wavelengths. Chemical measurements comprise measurements of pH, gases dissolved in the liquid, redox potential, etc.

Additionally, the measuring device 8 comprises an electronic circuit 17 connected to all the sensors and electronic elements, and comprising a microprocessor 171 (or microcontroller) configured to manage the acquisition of all the sensors and to perform pre-processing operations: filtering, averaging, counting, corrections, etc.

The measuring device 8 also comprises a transmission or communication module 19 for transmitting the observation data to the control device 11.

In the case of measuring device 8 embedded in a float 9 (FIGS. 6, 7) the measuring device may advantageously comprise a power management module 21 comprising a battery for powering all electronic elements of the measuring device 8. Additionally, the measuring device 8 may also comprise means which may be the same electrical measuring sensors for measuring the buoyancy level of the float 9.

In a first embodiment, the microprocessor 171 is configured to determine, at successive instants, vectors of physical variables of interest relating to the biochemical process according to the corresponding measurements performed at successive instants. Thus, the transmission or communication module 19 is configured to transmit to the control device 11 the observation data comprising vectors of physical variables determined by the microprocessor 171, as well as the corresponding measurements.

In another embodiment, the observation data transmitted by the measuring device 8 to the control device 11 comprise only the measurements made by the sensors 13, 15, 151. In this case, the control device 11 is configured to receive these measurements and to determine, at successive instants, the vectors of physical variables of interest relating to the biochemical process according to the corresponding measurements.

Each vector of physical variables of interest comprises a first temperature or heat transfer variable determined from the temperature difference between two points. The vector of physical variables of interest comprises at least one other physical variable corresponding to the measurement(s) other than temperature. This at least one other physical variable may be one of the second to twelfth variables of interest defined hereinabove (see also FIGS. 6 and 7).

The second variable relates to gas release. This may be determined by a local count of the number of bubbles, using a differential pressure sensor (as described in patent application FR039275) that may advantageously be integrated into the float 9, or by gas flow measurements measuring the total amount of gas released from the vessel.

The third variable relates to the density of the liquid determined by differential pressure measurements between two depths, using a pressure sensor (as described in said application FR039275) which may advantageously be integrated into the float 9, and/or from the measurements of buoyancy levels of the float 9.

The fourth variable relates to the electrical conductivity of the liquid determined from electrical measurements of voltages and/or currents at one or more frequencies and/or by resonance frequency measurements, using an electromagnetic sensor which may advantageously be integrated into the float 9.

The fifth variable relates to the permittivity of the liquid determined from electrical measurements preferably at several frequencies with a specific electrode or by resonance frequency measurements which may advantageously be integrated in the float 9.

The sixth variable relates to the movement of the liquid determined from acceleration measurements by means of an inertial unit or accelerometer which may advantageously be integrated into the float 9.

The seventh variable relates to the measurement of the PH of the liquid, determined from electrical measurements with a specific electrode which may advantageously be integrated into the float 9.

The eighth variable relates to the redox potential determined from electrical measurements with a specific electrode which may advantageously be integrated into the float 9.

The ninth variable relates to the dissolved oxygen determined from electrical measurements and/or optical measurements.

The tenth variable relates to the dissolved CO₂ determined from electrical measurements with a specific electrode and/or from optical measurements which may advantageously be integrated into the float 9.

The eleventh variable relates to the optical absorption spectrum (turbidity, colour) determined from optical measurements of at least two wavelengths.

The twelfth variable relates to the rotatory power determined from the optical measurements using polarised light which varies by the presence of chiral molecules.

The control device 11 is configured to record the initial conditions of the biochemical process, such as the composition of the liquid substrate 7 in the vessel 5, such as the concentration of all ingredients, such as sugar, characteristics of the juice, the yeast strains, or by chromatographic analyses etc. The control device 11 is also configured to collect results from processing, record them and compare them with thresholds set by the user according to his/her skills in the art and the specific features of the product he/she is seeking to obtain.

Additionally, the control device 11 is configured to control actuators (23) for regulating the biochemical process. Indeed, the control device 11 is configured to control the regulation of the biochemical reactor 3 by at least one action aₜ to be performed on the vessel 5. By way of example, the actions may comprise modification of the agitation speed (circulation, rotation, etc.) or activation of stirring if the gas flow rate is too low; modification of the temperature (increasing or decreasing the heating or cooling circuit set point), for example, decreasing the temperature set point if the variation in density is too great; modification of the oxygen supply rate; supply of nutrients (mineral salts, sugar, nitrogen, etc.) or other elements to activate or stabilise the biochemical process, such as supply of oxygen or nitrogen if the gas release slows down; supply of yeast or bacterial strains, etc.

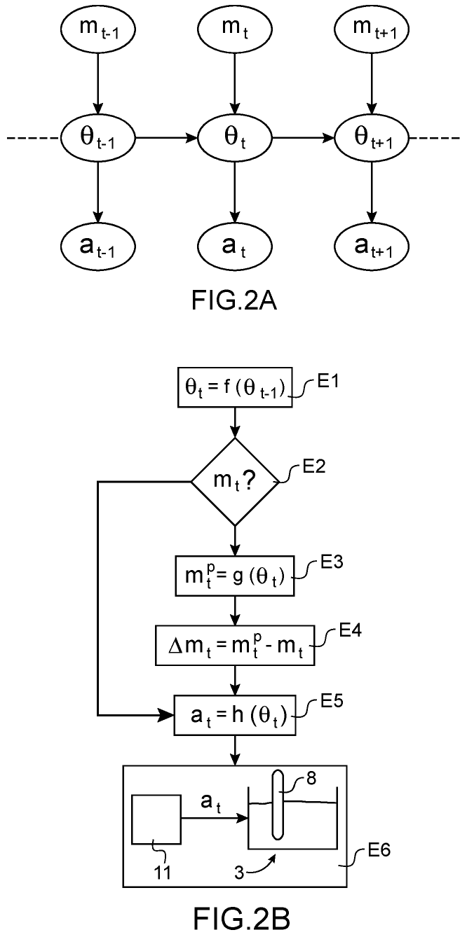

FIGS. 2A and 2B illustrate an algorithm for controlling the biochemical reactor, according to a preferred embodiment of the invention. More particularly, FIG. 2A represents the control algorithm in the form of a graph, while FIG. 2B represents it in the form of a flowchart.

This algorithm is based on the definition of three temporal functions defined over a period T corresponding to the biochemical process. The first function ƒ relates to the temporal prediction at a time instant t, of values of physical variables θₜ with full knowledge of their values at a previous time instant:

[Math. 1]

ƒ:θₜ₋₁→θₜ (1)

The second function g associates the measurements mₜ performed by the sensors with the physical variables at a given time instant:

[Math. 2]

g:θₜ→mₜ (2)

The third function h determines the action aₜ to be performed in the vessel according to values of physical variables at a given time instant t:

[Math. 3]

h:θₜ→aₜ (3)

The control algorithm is configured to determine a series of actions aₜ for each time step t, with full knowledge of the physical variables θₜ obtained from the measurements mₜ performed by the sensors.

FIG. 2A is a directed graph organized in matrix form with three rows and T columns, where each column represents a time step t ranging from 1 to T. The first row corresponds to a first time sequence of nodes representative of the measurements mₜ, while the second row corresponds to a second time sequence of nodes representative of vectors of the physical variables θₜ, and the third row corresponds to a third time sequence of nodes representative of vectors of regulation actions aₜ. The nodes in each column are connected by arrows pointing from the first row to the second row and then from the second row to the third row. Additionally, the nodes in the second row (i.e. the vectors of physical variables θₜ) are also connected by arrows pointing sequentially in the increasing time direction from 1 to T.

More particularly, on the sequential arrows of the second row, the control device 11 is configured to predict, at successive instants: predictive vectors of physical variables according to vectors of previous physical variables derived from measurements on the one hand, and anticipative measurements according to predictive vectors of physical variables on the other hand.

Additionally, on the arrows pointing from the first row to the second row, the control device 11 is configured to calculate, at successive instants, measurement discrepancies between anticipative measurements and corresponding actual measurements. The control device 11 is then configured to correct the vectors of physical variables at successive times according to the corresponding measurement discrepancies.

On the third row, the control device 11 is configured to determine, at successive instants, vectors of regulation actions according to the corresponding vectors of physical variables. This allows the control device 11 to control the regulation of the biochemical reactor 3 by triggering, at successive instants, regulation actions based on the vectors of regulation action.

FIG. 2B is a flow chart illustrating the steps for controlling the biochemical reactor at any instant t∈[1,T] during the biochemical process according to the directed graph of FIG. 2A

In step E1, the control device is configured to perform a prediction of each predictive vector of physical variables of the biochemical process using the first time function ƒ defining values of physical variables at instant t with full knowledge of their values at the previous time instant t−1:

[Math. 4]

θₜ=ƒ(θₜ₋₁) (4)

This first function ƒ is predetermined, for instance, by a standard Markov model of the order of at least 1.

Step E2 is a test to check whether the sensors are functional at the instant t, i.e. whether measurements mₜ performed by the sensors are present at the instant t. If yes, then proceed to Step E3. Otherwise proceed to Step E5.

Step E3 relates to the prediction of each anticipative measurement using the second function g associating measurements performed by the measuring device 8 at the time instant t with the vector of physical variables calculated in Step E1:

[Math. 5]

mₜ^{p}=g(θₜ) (5)

The second function g may be predetermined by equations correlating measurements performed by the measuring device 8 to the physical variables.

Step E4 relates to a correction of the vector of physical variables θₜ by inversion (or pseudo-inversion) of the second function g, and calculation of the discrepancy Δmₜ between the actual measurements mₜ performed by the float and those mₜ^{p} predicted:

[Math. 6]

Δmₜ=mₜ^{p}−mₜ (6)

Step E5 relates to the determination of each vector of regulation action using the third function h defining a correspondence at each time instant to between actions to be performed and values of physical variables:

[Math. 7]

aₜ=h(θₜ) (7)

The third function h may be predetermined for instance by a user skilled in the art by preselecting thresholds on physical variables for triggering actions.

In Step 6, the control device 11 is configured to control the regulation of the biochemical reactor 3 by triggering, at said successive instants, regulation actions based on said vectors of regulation action.

Advantageously, it should be noted that in the event of measurement failure, the control device is configured to continue performing Steps E1, E5 and E6 separately from measurements. Thus, vectors of regulation action can be determined according to predictive vectors of physical variables in the event of failure of the measuring device 8.

This control operation may take the form of Kalman filtering if the functions ƒ and g are linear [Welch95], or alternatively, of stochastic particle filtering in any other case [Doucet09]. Advantageously, it does not require a prior database.

It should be noted that the event where at least one of the three functions is unknown, it can be learned automatically with full knowledge of a complete time series (i.e. t=1 to T) of a biochemical reaction associating measurements m_{1:T} and actions a_{1:T} performed in the vessel 5.

Indeed, a database can be built during a regulation learning phase comprising several sets {m_{1:T}, a_{1:T}} of correspondence data between measurements m_{1:T} performed by the measuring device 8 and actions a_{1:T} performed in the vessel 5. The correspondence data are generated automatically by a learning process. Indeed, the physical variables θ_{1:T} become hidden variables that are estimated automatically by the learning process, and it is not necessary to known them as long as m_{1:T} and a_{1:T} are known. The actions are performed during the regulation learning phase of the biochemical process by an operator skilled in the art.

Thus, at least any one of the first, second and third functions can be determined based on correspondence data {m_{1:T}, a_{1:T}} acquired from the database. Such determination of functions can be performed using an algorithm of the Expectation Maximisation (EM) type [Roweis99] where they are assumed to be linear.

In the event where the functions are not linear, they can be determined based on correspondence data {m_{1:T}, a_{1:T}} using a recurrent neural network (RNR) [Goodfellow16], the same network itself serving as a control algorithm are the end of its training.

Once determined, these functions can be used to control any biochemical reactions according to the algorithmic model previously explained.

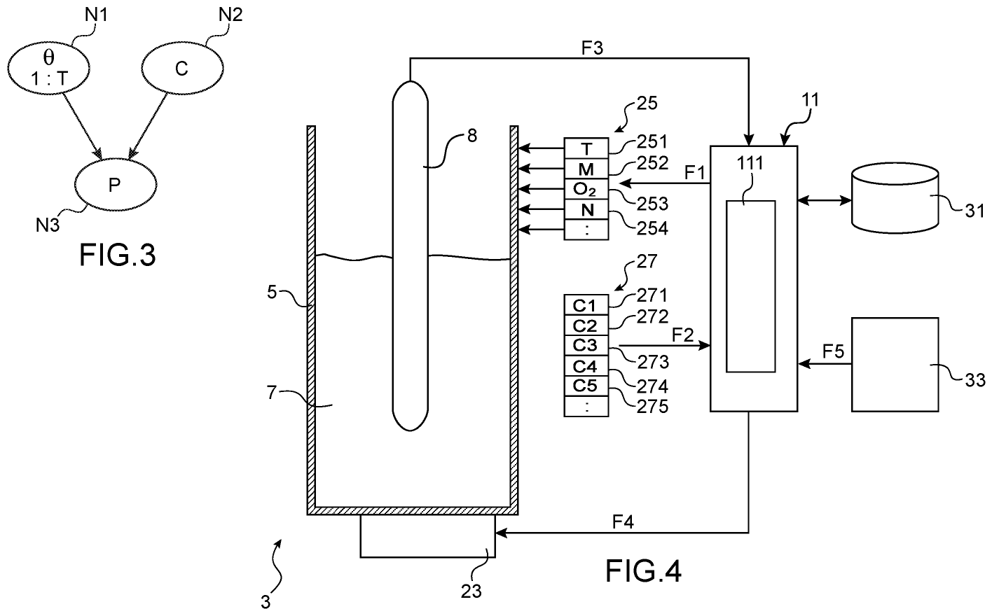

FIG. 3 is a directed graph representing a property learning algorithm of the compound at the outlet of the biochemical reactor, according to a preferred embodiment of the invention.

This graph illustrates how the control device 11 can be used to build a property learning model for the compound at the outlet of the biochemical reactor 3. The graph comprises a first node N1 representing a vector of physical variables θ_{1:T}, a second node N2 representing a vector of characteristics c of the elements reacted in the vessel 5, and a third node N3 representing a vector of properties p of the product at the outlet. The property learning model allows each of the first θ_{1:T} and second c nodes to be connected to the third node p by a directed arrow.

Indeed, at the end of the biochemical process, the compound obtained has different properties that depend on the evolution of vectors of physical properties θ_{1:T} during the processing in the vessel 5, as well as of certain characteristics c of elements reacted in the vessel 5.

These characteristics form a vector of characteristics c comprising, for instance, the nature of microorganisms, characteristics of raw materials and, possibly, of added nutrients. The nature of the microorganisms comprises, for instance, the type of yeast, bacteria, microalgae and their quantity. The characteristics of the raw material comprise, for instance, in the case of wine, sugar concentration, grape variety, terroir, weather, harvest and pressing data. In other applications, these raw material characteristics may include the nature of the wastewater for treatment or the bacterial culture substrate for bioreactors. Added nutrients comprise, for instance, yeast activators, mineral salts, or other elements such as activated carbon, glucose, etc.

Thus, the control device 11 builds a property learning database comprising a collection of sets {θ_{1:T}, c} between vectors of physical variables θ_{1:T} and vectors of corresponding characteristics c.

Additionally, the properties derived from the biochemical process form a vector of properties p indicative of the quality of the compound. This can be assesses using different devices, such as those of the type that cannot be integrated for in-situ measurement. By way of example, the concentration of the different metabolites can be determined in laboratory with off-line equipment (chromatography, IR spectroscopy, etc.). The presence and concentration of microorganisms can be determined by culture and biological analyses (PCR). Other analyses, for instance relating to organoleptic observations (taste, colour, fragrance, etc.) of the final product can be performed by persons skilled in the art.

The control device 11 thus records all observations and results of on-line or off-line analyses forming the final product in the form of vectors of properties p of the compound at the outlet. Thus, after a set of biochemical processes, the property learning model comprises a collection of sets of vectors of property p of the compound at the outlet.

Additionally, the control device 11 is configured to build correlations between vectors of physical variables θ_{1:T} recorded at the end of the biochemical process and the corresponding vectors of characteristics c in order to obtain vectors of properties p of the compound at the outlet. These correlations between vectors of physical variables and corresponding vectors of characteristics are determined by estimation of a learning function z:

[Math. 8]

z:θ_{1:T}×c→p (8)

With full knowledge of at least a set {θ_{1:T}, c} of the collection of sets in the database, the learning function z can be estimated using different statistical models, such as polynomial regression, or kernel models, or neural networks, etc. [Bishop06]. The estimation of the learning function z will be even better the larger the database.

The learning function z helps to understand the influence of characteristics of reacted elements, or of evolution of the biochemical process in the properties obtained on the compound. Thus, it becomes possible to adapt the characteristics of the elements placed in the vessel 5 and to regulate the evolution of physical variables of biochemical reactions using the control algorithm, in order to obtain specific properties of the compound at the outlet of the reactor 3. Indeed, with full knowledge of the correlation between the physical variables θ_{1:T} and the characteristics c that confer the properties p, the control device can specify during the biochemical process a series of actions a_{1:T} to “force” the physical variables θ_{1:T} to follow a time profile, thus ensuring that the product has the desired properties at the outlet.

FIG. 4 very schematically illustrates a system and a method for in-situ monitoring of a biochemical fermentation process in a reactor, according to a preferred embodiment of the invention.

More particularly, FIG. 4 illustrates an embodiment of the method for monitoring a fermentation process in the field of winemaking, according to the invention.

The biochemical reactor 3 comprises a vessel 5 intended to receive a juice 7 on which a fermentation process has to be performed to make wine. As illustrated on FIG. 1, the monitoring system comprises a measuring device 8 (that can be integrated into a float) and a control device 11.

The measuring device 8 is configured to be partially immersed in the juice 7 contained by the vessel 5, while performing, at successive instants, measurements of physical quantities and, more particularly, measurements of temperature, pressure, acceleration and frequency.

Advantageously, the measuring device 8 is configured to process measurement data in order to calculate physical variables of interest, such as density, CO₂ flow rate, conductivity, and movement of the liquid, before transmitting to the control device 11 observation data comprising the physical variables and corresponding measurements.

Alternatively, the measuring device 8 may be configured to transmit to the control device 11 observation data comprising only the corresponding measurements. In this case, it is the control device 11 that processes the measurement data to calculate the physical variables of interest. In both cases, the physical variables form indicators of interest that will allow the control device 11 to automatically perform direct feedback on the fermentation process.

The control device 11 allows an IHM 111 to be used to capture the recipe 25 to be applied to the method. The recipe may include the temperature profile 251, conditions for activation of stirring 252, oxygenation conditions 253, conditions for activating nutrient supply 254, etc.

The recipe 25 comprises a set of threshold parameters and coefficients logically combined to perform setpoints or actions, as in the non-limiting examples below.

In a first example: If (Density<Threshold x) or if (Gas flow<Threshold y) then: Stirring action according to a stirring speed=Coef. b*(Threshold y−Gas flow rate).

In a second example: If (Density>Threshold w) and if (Gas flow<Threshold z) then: Add oxygen according to an Oxygen flow rate=Coef o*(Threshold z−Gas flow rate).

In a third example: Temperature setpoint=Density*Coef k+Threshold l. Additionally, the control device 11 is used to capture input characteristics 27 of the juice 7, which may be derived from additional analyses (on-line or off-line) or from the winemaker skilled in the art. These input characteristics comprise, for instance, the grape variety 271, the terroir 272, the oenological recipe 273, the vintage 274, the yeast strain 275, etc. It should be noted that the characteristics are input data for determining the recipe 25 to be applied to the method process based on skills in the art.

Additionally, and if necessary, certain measurements can be performed off-line from time to time, and captured by the user or transmitted directly to the control device.

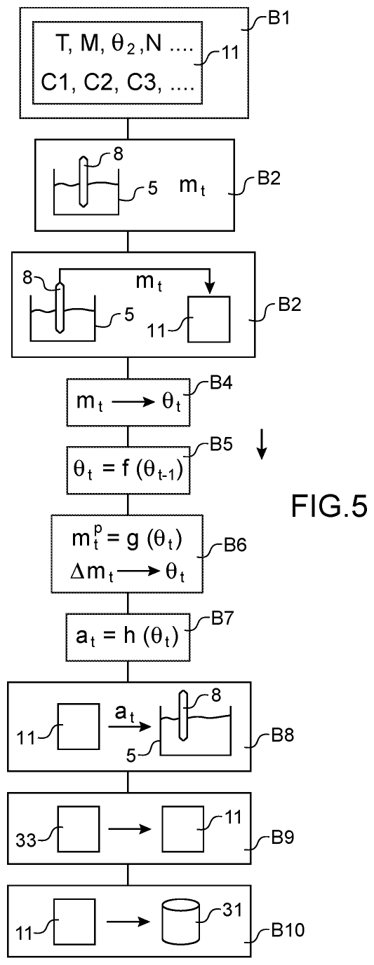

FIG. 5 very schematically illustrates a method for in-situ monitoring of the biochemical fermentation process in relation to the system on FIG. 4.

Block B1 relates to the initialisation during which the control device 11 acquires the recipe to be applied (arrow F1) to the fermentation process. This recipe can be captured by the operator or determined automatically from the input characteristics 27 (arrow F2) relating to the grape variety 271, the terroir 272, the oenological recipe 273, the vintage 274, and the yeast strain 275.

In block B2, the measuring device 8 measures temperatures, pressures, accelerations, frequencies, etc. at each time step t.

At block B3, the measuring device 8 transmits to the control device 11 (arrow F3) at each time step t, observation data comprising the measurements mₜ performed.

Optionally, the control device 11 is also configured to acquire further measurements performed off-line from time to time, for instance.

In block B4, the control device 11 calculates physical variables θₜ from corresponding measurement data mₜ thus forming vectors of physical variables. A vector of physical variables may comprise temperature (or heat transfer), density, agitation, CO₂ flow rate, acidity, sugar concentration, conductivity, etc. These parameters of interest allow the control device 11 to determine immediate feedback by controlling (arrow F4) the actuators 23 for regulating the biochemical process.

In blocks B5-B8, the control device 11 performs the steps described in relation with FIG. 2B, at each time step t.

In block B5, the control device 11 predicts a predictive vector of physical variables using the first function f according to the equation (4).

In block B6, the control device 11 predicts anticipative measures using the second function g according to the equation (5), calculates discrepancies between actual measures taken by the measuring device 8 and those predicted according to the equation (6), and uses them to correct the vector of physical variables θₜ.

In block B7, the control device 11 calculates the vector of regulation action using the third function h, according to the equation (7), and displays the values of parameters useful for regulation.

In block B8, the control device 11 controls the regulation of the fermentation process by triggering regulation actions based on the vector of regulation action, as well as the recipe and specific instructions for the process.

These actions allow for prompt intervention in the fermentation process in order to regulate parameters according to the recipe and/or to prevent and avoid anomalies, such as “fermentation stoppage” if the yeasts or bacteria no longer act.

A first example of regulation action can be an increase or decrease in the temperature of the juice 7 according to the temperature profile 251 specified initially. Indeed, the development of bacteria depends mainly on temperature, and this affects the aromas of the wine, among other things. Thus, the temperature profile is defined at the beginning by the oenologist or wine expert according to the type of wine and the desired aroma.

A second regulation action may be stirring 252 and stirring speed depending on the density and/or the gas flow rate. A third regulation action may be oxygenation 253 and the quantity of oxygenation depending on the gas flow rate. A fourth action may be addition of yeast 254 depending on the temperature, and/or the density, and/or the gas flow rate, etc.

Moreover, the control device 11 is advantageously configured to create output databases 31 which will allow algorithmic learning on the basis of organoleptic appreciation, such as evaluation of quality of the product with regard to the desired aromatic profile.

Indeed, at block B9, at the end of the fermentation process, the control device 11 records (arrow F5) the properties 33 of the final product (for instance, the evaluation and annotation of the final product made by the oenologist).

In block B10, the control device 11 constructs a property learning database 31 of the final product gathering the characteristics of the liquid at the beginning of the process, the parameters of the recipe, the recording of measurements and corresponding physical variables, additional analyses (on line or off line), and evaluations and properties P of the quality of the final product.

Thus, by providing recordings at the end of each new fermentation process, the property learning database 31 contains a collection of physical variables θ_{1:T}, corresponding characteristics c, and evaluations and properties p of the final product at the output of each process. This allows for automated feedback by using various statistical models to determine a correlation or a learning function z associating the properties p of the final product according to physical variables θ_{1:T} and the corresponding characteristics c, according to the equation (8). This helps to optimise all control parameters of the process, such as thresholds and coefficients of the recipe in order to obtain the best results.

FIG. 6 very schematically illustrates a system 1 for in-situ monitoring of a biochemical process in a biochemical reactor 3, according to one preferred embodiment of the invention.

This embodiment is similar to that of FIG. 1 save for the fact that the measuring device 8 is advantageously integrated into a float 9 intended to be inserted by floating partially immersed in the liquid 7 contained in the vessel 5. The float 9 helps to offset any variability in the filling levels of the vessel 5. This embodiment is advantageous for large vessels, such as those used to make fermented beverages.

FIG. 7 very schematically illustrates an instrumented float, according to a preferred embodiment of the invention.

The float 9 with a predetermined mass according to the desired measuring range, is fitted with a stem 91 extending upwards and a plunger 92 intended to keep it partially immersed and fully vertical in the liquid 7 when it is inserted into the vessel 5.

The measuring device is integrated into the float 9, such that the latter comprises an electronic circuit 17, one or more temperature probes 13a, 13b, mechanical, electrical and possibly optical measurement sensors 15, as well as a communication module 19 and a power management module 21.

The mechanical measurement sensors comprise a differential pressure sensor 151 and a movement sensor 152. The electrical measurement sensors comprise at least two electromagnetic sensors 153a, 153b and possibly sensors 154 for measuring pH, measuring gases dissolved in the liquid, measuring redox potential, etc. Optional optical measurement sensors 155 may comprise light intensity measurement sensors.

The electronic circuit 17 is connected to all the sensors 13, 15 and electronic elements 17, 21 integrated in the float 9, and comprises an analogue-digital converter, a memory and a microcontroller or microprocessor 171 intended to manage the acquisition of all the sensors.

The temperature sensors 13a, 13b integrated in the float 9 are advantageously shifted to the top and bottom of the broad part of the float 9, in order to measure any temperature gradient.

The differential pressure sensor 151 is arranged on the lower part of the float 9 and comprises two connected tubes 151a, 151b of different lengths, as well as a piezoelectric membrane (not illustrated) which deforms under the effect of a pressure difference generating a differential voltage. The free ends of the two tubes 151a, 151b are configured to be immersed in the liquid 7 at two different depths, thus creating in the circuit of the pressure sensor 151 a differential voltage representative of the differential pressure between the two different depths. The microprocessor 171 of the electronic circuit 17 is configured to determine the density of the liquid 7 according to the differential pressure.

Additionally, the generation of gas bubbles at the ends of the tubes 151a, 151b influences the differential pressure measurement. Specifically, when the bubbles fall off, the sensor 151 generates voltage spikes representative of the occurrence of bubbles, such that each spike coincides with the occurrence of a gas bubble at the end of one of the two tubes 151a, 151b. The microprocessor 171 of the electronic circuit 17 is configured to count the number of bubbles, in order to determine the flow rate of the gas (typically CO₂). This is set out in detail in the applicant's patent application FR3039275.

Thus, the differential pressure sensor 151 is used to quantify gas release from the reaction in vessel 5 and determine the density of the liquid 7. However, initially, when the float 9 is placed in the vessel 5, some of the liquid rises in the tubes 151a, 151b before filling up with gas and, therefore, no accurate measurement of density can be performed during this transient state. In contrast, in the steady state, the tubes 151a, 151b are filled with gas and therefore the differential pressure sensor 151 delivers a very accurate measurement of the liquid 7 density.

Advantageously, the electromagnetic sensors 153a, 153b are not affected by this phenomenon and deliver an accurate measurement of the density both in transient state and steady state. Thus, the float 9 comprises at least the first 153a and second 153b electromagnetic sensors coupled to the electronic circuit 17. Each electromagnetic sensor 153a, 153b comprises an LC inductive-capacitive circuit creating a resonator with a natural resonant frequency that depends on the fluidic environment of the resonator.

More particularly, each of the sensors 153a, 153b comprises a resonator which comprises an active reactance in the form of electrode(s), so-called reactance electrode(s) associated with a corresponding passive coupling element. The reactance electrode can be either capacitive or inductive. In particular, if the electrode is capacitive, then the corresponding coupling element will be inductive and vice versa, thus forming a resonator with a resonance frequency that varies according to the buoyancy level of the float 9 and the nature of the liquid 7 contained in the vessel 5.

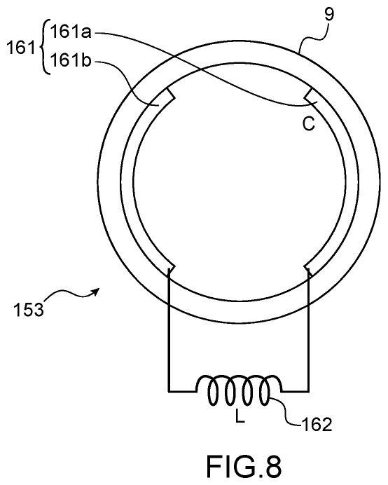

FIG. 8 very schematically illustrates the arrangement of an electromagnetic capacitive sensor on the side surface of the float, according to the embodiment on FIG. 7.

by way of example, let us consider the case of an electromagnetic sensor 153 with a capacitive reactance electrode. The reactance electrode (161) then comprises two conductive armatures 161a and 161b arranged opposite each other on the inner surface of the float 9, matching the cylindrical shape of this surface. Thus, a tubular capacitor is formed by the two armatures 161a and 161b. Since the geometry of the capacitor is invariant, its capacity C depends essentially on the nature and level of the liquid 7 contained in the vessel 5. It should be noted that, alternatively, the conductive armatures 161a and 161b of the sensor can be interdigitated.

Additionally, the armatures 161a and 161b of the capacitance capacitor C are connected to an inductance which may be a coil 162 of predetermined inductance L, thus forming a resonance circuit with a frequency that depends on the capacitance C which may be variable and the inductance L which is constant. The electromagnetic sensor 153 measures the resonance frequency thus allowing the microprocessor 171 to deduce the value of the capacitance C and consequently, the permittivity of the liquid at the armatures 161a and 161b. Thus, the resonance frequency varies according to the level (i.e. liquid/air interface) of the liquid 7 in the vessel 5 and its nature. The measurement by an electromagnetic sensor 153 of this resonance frequency thus provides information on the nature and configuration of this environment.

The first electromagnetic sensor 153a is integrated into the plunger, so that when the float 9 is inserted into the liquid 7, the active electrode of the first electromagnetic sensor 153a is in the fully immersed part. Thus, there is no air-liquid interface at the armatures 161 and therefore, the measured resonant frequency is indicative only of the nature of the liquid 7. The first electromagnetic sensor 153a is thus configured to perform a reference measurement relating to physical properties of the liquid 7 and more particularly, to electrical properties such as conductivity and permittivity of the liquid 7 contained in the vessel 5.

The second electromagnetic sensor 153b is integrated into the stem 91 of the float 9. In particular, the active electrode of the second sensor 153b is arranged along the entire length of the stem 91. Thus, when the float 9 is inserted into the liquid 7, the active electrode always comprises a part partially immersed in the liquid. The second sensor 153b then measures the immersion level of the stem 91 and hence the volume of the displaced liquid indicative of the density of the liquid 7. Indeed, the variation in resonance frequencies is indicative of the variation in the permittivity of the insulation between the armatures due to displacement of the air-liquid interface. It should be noted that the relative permittivity of a liquid to air is quite substantial. In particular, the insertion of the float 9 into the liquid 7 increases the level of the liquid, which thereby increases the capacitance thus generating a decrease in the resonant frequency.

The microprocessor 171 is configured to calculate the density of the liquid 7 based on the measurement of the volume of the displaced liquid performed by the second sensor 153b and the measurement of the electrical properties of the liquid performed by the first sensor 153a.

Indeed, with full knowledge of the mass of the float 9, the microprocessor 171 can estimate the value of the density of the liquid 7 from the variation in level of the liquid which is proportional to the volume of the liquid displaced by the float 9. Additionally, the measurement of the electrical properties of the liquid 7 performed by the first electromagnetic sensor 153a is a reference measurement that allows the microprocessor 171 to correct the first estimate and determine the density of the liquid 7 with greater accuracy.

Thus, the electromagnetic sensors 153a, 153b provide an accurate measurement of the density of the liquid 7 thereby circumventing the inaccurate measurement of the differential pressure sensor 151 at the beginning of the reaction. In addition, having two independent density measurements helps to enhance the accuracy of the steady-state measurement and possibly detect any sensor malfunction.

The movement sensor 152 is, for instance, an inertial unit or a three-axis accelerometer which may be integrated into the immersed part 92 of the float 9. This movement sensor 152 is configured to measure the movement of the float 9 and indirectly that of the liquid 7, and to convey this indirect information on movements of the liquid to the microprocessor 171. Indeed, the liquid 7 may be subjected to a controlled stirring movement. The microprocessor 171 collects the measurements provided by the movement sensor 152 in order to calculate the movement of the liquid 7. By measuring the movement of the liquid 7 when subjected to controlled stirring, its viscosity can be estimated. Viscosity estimation could also be considered either by applying a rotation (e.g. with an agitator) or by moving the float 9 away from its equilibrium position and measuring the time taken to return to it.

Advantageously, the movement sensor 152 is further configured to validate or correct the acquisition depending on whether the liquid 7 is at rest or in motion. It also allows the elimination of artefacts related to external disturbances.

The float 9 may also include other sensors 154 adapted to measure chemical properties of the liquid. By way of example, the float 9 may comprise a sensor for measuring pH, sensors for measuring gases (O₂, CO₂) dissolved in the liquid, a sensor for measuring redox potential, a sensor for measuring the concentration of specific molecules by the ISE (ion selective electrode) technique, etc. Advantageously, the sensors 154 for measuring chemical properties can be integrated into the float 9 using DLC (diamond-like carbon) electrodes, which allows these sensors to be miniaturised. Their selection and use will depend on the needs of the application and the aggressiveness of the environment.

It should be noted that the acquisition frequency of each sensor can be defined according to the specificities of each measurement (e.g., greater than 1 Hz for differential pressure measurement, less than 0.1 Hz for density measurement using the electromagnetic method, etc.).

The wireless communication module 19 comprises an antenna 20 integrated into the stem 91 of the float 9 and connected to the various sensors 13, 15 via the electronic circuit 17. The antenna 20 is configured to transmit the data relating to measurements performed by the sensors to the control device 11 according to a wireless communication system (e.g., NFC, RFID, Blue tough, Wifi . . . ) with low energy. Thus, the data are communicated by the emerging part of the float 9 to the control device 11, so that it can control the various actions to be performed in order to regulate the biochemical process.

The power management module 21 comprises a battery or other power supply means advantageously integrated into the plunger 92 of the float 9. Depending on the use, the autonomy required for the minimum duration of an acquisition will determine how the power supply means will be sized. In the case where the power supply is a battery, the latter is preferably arranged at the bottom to serve as ballast. In contrast, a remote power supply would preferably be located in the non-immersed part.

Advantageously, the monitoring system 1 comprises a calibration and inductive recharging module 31 intended to calibrate the float 9 and to remotely recharge the battery integrated into the float 9. It should be noted that, depending on the nature of the vessel 5 and the liquid 7, the float 9 can be remotely and continuously powered. The calibration and inductive recharging module 31 can advantageously be comprised in a protective case 33 for the float 9.

The microprocessor 171 of the electronic circuit 17 in relation with the analogue-to-digital converter and the memory is configured to manage the acquisition of all the sensors 13, 15, and to perform a pre-processing: of filtering, averaging, counting and corrections, thus allowing that only the useful part of this information is transmitted. By way of example, the microprocessor 171 can apply corrective factors to measurements of temperature and pressure, for instance, by disregarding measurements in the event of agitation, or by applying corrective factors to the measured values if the sensors are sensitive to temperature and pressure conditions.

Additionally, the microprocessor 171 is configured to merge all measurement data in order to detect any anomalies arising from, for instance, deterioration or fouling of a sensor. It is configured to issue a preventive maintenance alert. The microprocessor 171 is also adapted to measure the system's response when the liquid is subjected to an external action: agitation, modification of the temperature set point, supply of nutrients, yeast, oxygenation, etc.

The microprocessor 171 is also configured to process measurements, in order to extract the physical variables characterising the liquid 7, which are necessary for controlling the biochemical process in the vessel 5.

Thus, the electronic circuit 7 manages acquisition of all the sensors, extracts useful data and formats same before transmitting same to the control device 11.

Needless to say that a person skilled in the art can make various modifications to the invention herein described, by way of non-limiting examples only.

### BIBLIOGRAPHY

[Welch95]: An introduction to the Kalman filter, G. Welch, G. Bishop, technical report 2006.
[Doucet09]: A Tutorial on Particle Filtering and Smoothing: Fifteen years later, A. Doucet, A. M. Johansen, technical report 2012.
[Roweis99]: A unifying review of linear Gaussian models, S. Roweis, Z. Ghahramani, Neural Computation 1999.
[Goodfellow16]: Deep learning, I. Goodfellow, Y. Bengio, A. Courville, Y Bengio, MIT press 2016.
[Bishop06]: Pattern recognition and machine learning, C. M. Bishop, Springer 2006.

1. A system for in-situ monitoring of a biochemical process in a reactor comprising a vessel (5) for receiving a liquid (7), characterised in that it comprises:
a measuring device (8) intended to be inserted into said vessel (5), said measuring device (8) being instrumented with sensors configured to take measurements relating to the biochemical process at successive instants and to transmit, at said successive instants, observation data representing at least the temperature and the density of the liquid, and
a control device (11) configured to control the regulation of the biochemical reactor (3) at said successive instants, according to said observation data received from the measuring device (8).

2. The system according to claim 1, characterised in that said measurements comprise measurements of temperature of the liquid and at least one other type of measurements among the following measurements: mechanical measurements of pressures and/or gas flow rate, and/or accelerations and/or buoyancy level, electrical measurements of voltages and/or currents and/or resonance frequencies, and optical measurements.

3. The system according to claim 1, characterised in that the measuring device comprises a microprocessor (171) configured to determine, at said successive instants, vectors of physical variables relating to the biochemical process according to the corresponding measurements made at said successive instants, the observation data transmitted by the measuring device (8) to said control device (11) comprising said vectors of physical variables and said corresponding measurements.

4. The system according to claim 1, characterised in that the observation data transmitted by the measuring device (9) to the control device (11) comprises said measurements, and in that the control device is configured to determine, at said successive instants, vectors of physical variables relating to the biochemical process according to the corresponding measurements.

5. The system according to claim 3, characterised in that each of said vectors of physical variables comprises a temperature variable, a liquid density variable determined from measurements of buoyancy levels and/or pressures, and at least one other variable from among the following variables: gas release determined from pressure measurements, electrical conductivity and/or permittivity of the liquid determined from electrical measurements, movement of the liquid determined from acceleration measurements, PH and/or redox potential determined from electrical measurements, dissolved oxygen and/or CO₂ determined from electrical measurements and/or optical measurements, optical absorption spectrum and/or rotatory power determined from optical measurements.

6. The system according to claim 1, characterised in that the control device (11) is configured to control regulation of the biochemical reactor by at least one of the following actions: modification of the agitation speed, modification of the temperature, modification of the rate of oxygen supply, nutrient supply or other elements for activating or stabilising the biochemical process, supply of yeasts or bacterial strains.

7. The system according to claim 4, characterised in the control device (11) is configured to:
predict, at said successive instants, predictive vectors of physical variables according to said previous vectors of physical variables derived from the measurements;
predict, at said successive instants, anticipative measurements according to said predictive vectors of physical variables;
calculate, at said successive instants, measurement discrepancies between the anticipative measurements and the corresponding actual measurements;
correct, at said successive instants, vectors of physical variables according to said measurement discrepancies;
determine, at said successive instants, vectors of regulation actions according to said vectors of corresponding physical variables; and
control regulation of the biochemical reactor by triggering, at said successive instants, regulation actions based on said vectors of regulation actions.

8. The system according to claim 7, characterised in that the control device (11) is configured to determine the vectors of regulation actions according to the predictive vectors of physical variables in the event of measurement failure by the measuring device (8).

9. The system according to claim 7, characterised in the control device (11) is configured to:
predict each predictive vector of physical variables using a first temporal function f defining values of physical variables with full knowledge of their values at a previous time instant, said first function ƒ being predetermined by a standard Markov model of the order at least 1;
predict each anticipative measurement using a second function g associating the measurements performed by the measuring device with the physical variables at a given time instant, said second function g being predetermined by equations correlating the measurements with the physical variables, and
determine each vector of control actions using a third function h defining a correspondence between the actions to be carried out and the values of the physical variables at a given time instant, said third function h being predetermined by a pre-selection of action triggering thresholds.

10. The system according to claim 9, characterised in that it comprises a database built during a regulation learning phase comprising correspondence data between measurements m_{1:T} performed by the measuring device and actions a_{1:T} performed in the vessel, said correspondence data being generated automatically by a learning process, and in that at least one of the first, second and third functions is determined from said correspondence data acquired from said database.

11. The system according to claim 1, characterised in that the control device (11) is further configured to build a property learning model defining correlations associating properties p of the final product according to the corresponding physical θ_{1:T} and characteristic c variables.

12. The system according to claim 11, characterised in that the control device (11) is configured to determine said correlations by estimating a learning function using a statistical model of the polynomial regression type, or kernel model, or neural network.

13. The system according to claim 1, characterised in that the measuring device comprises at least one temperature probe (13a, 13b), and a differential pressure sensor (151) comprising two connected tubes (151a, 151b) of different lengths.

14. The system according to claim 1, characterised in that the measuring device is integrated into a float (9) comprising:
at least a temperature probe (13a, 13b);
a differential pressure sensor (151) arranged on the lower part of the float (9) and comprising two connected tubes (151a, 151b) of different lengths;
at least two electromagnetic sensors (153a, 153b) comprising armatures mounted on the walls of the float;
a movement sensor (152) configured to measure movements of the liquid;
a wireless communication module (19) comprising an antenna (20) arranged on the upper part of the float, said communication module (19) intended to transmit data relating to measurements performed by the sensors;
a power management module (21) comprising power supply means arranged in the lower part of the float; and
an electronic circuit (17) connected to all the sensors and electronic elements integrated into the float, said electronic circuit comprising a microprocessor (171) intended to manage the acquisition of all the sensors and transmission of data.

15. The system according to claim 14, characterised in that it comprises a calibration and inductive recharging module (31) intended to calibrate the float (9) and to remotely recharge the power supply means integrated into the float, said calibration and inductive recharging module (31) being comprised in a protective case (33) of the float.

16. A method for in-situ monitoring of a biochemical process in a reactor comprising a vessel (5) for receiving a liquid (7), characterised in that it comprises the following steps:
take measurements relating to the biochemical process at successive instants;
transmit, at said successive instants, observation data representing at least the temperature and the density of the liquid, and
control, at said successive instants, regulation of the biochemical reactor according to said observation data.
